(19) United States
(12) Patent Application Publication
(10) Pub. No.: US 2025/0182866 A1
(43) Pub. Date: Jun. 5, 2025

(54) SYSTEM FOR PATIENT DATA MANAGEMENT

(71) Applicant: Proxwell LLC, Philadelphia, PA (US)

(72) Inventors: Lawrence Choi, Philadelphia, PA (US); Jeffrey R. Lunger, Philadelphia, PA (US); John P. McLeod, Philadelphia, PA (US)

(73) Assignee: Proxwell LLC, Philadelphia, PA (US)

(21) Appl. No.: 18/964,980

(22) Filed: Dec. 2, 2024

Related U.S. Application Data
(60) Provisional application No. 63/605,149, filed on Dec. 1, 2023.

(51) Int. Cl. G16H 10/60; G06F 21/33; G16H 40/20; G16H 40/67; G16H 80/00

(52) U.S. Cl. CPC G16H 10/60; G06F 21/33; G16H 40/20; G16H 40/67; G16H 80/00

(57) ABSTRACT

There may be one or more methods, systems, and devices for data management as it relates to patient care in the home or non-traditional setting. Additionally, the patient care team may be managed by one or algorithms of an application. Additionally, one or more sensors may connect to a device running a client instance of the application, which may be connected to one or more sensors that may be utilized to ensure accurate and up to date information is recorded regarding the patient.

### BACKGROUND

Within the field of home health care workers, there is often times situations where recording information on a physical piece of paper may incur labor intensive secondary steps to ensure everyone involved in the patients' care is up to date on the latest information regarding the patient. Accordingly, there is a need for novel approaches and improvements to existing approaches.

### SUMMARY

There may be one or more methods, systems, and devices for data management as it relates to patient care in the home or non-traditional setting. Additionally, the patient care team may be managed by one or algorithms of an application. Additionally, one or more sensors may connect to a device running a client instance of the application, which may be connected to one or more sensors that may be utilized to ensure accurate and up to date information is recorded regarding the patient.

### BRIEF DESCRIPTION OF THE DRAWINGS

A more detailed understanding may be had from the following description, given by way of example in conjunction with the accompanying drawings, wherein like reference numerals in the FIGS. indicate like elements, and wherein:

FIG. 1 Illustrates task tax taxonomy.

### DETAILED DESCRIPTION

The Appendix document that accompanies this description is intended to be fully incorporated by reference. Further, one or more techniques disclosed herein may be used in conjunction with one or more techniques disclosed in the Appendix.

In the following description, for purposes of explanation and not limitation, specific details are set forth such as particular structures, architectures, interfaces, techniques, etc. in order to provide a thorough understanding of the various aspects of various embodiments. However, it will be apparent to those skilled in the art having the benefit of the present disclosure that the various aspects of the various embodiments may be practiced in other examples that depart from these specific details. In certain instances, descriptions of well-known devices, circuits, and methods are omitted so as not to obscure the description of the various embodiments with unnecessary detail.

Further, various operations are described as multiple discrete operations, in turn, in a manner that is most helpful in understanding the illustrative embodiments: however, the order of description should not be construed as to imply that these operations are necessarily order dependent. In particular, these operations need not be performed in the order of presentation, and may be performed in any order, as well as including or excluding one or more disclosed operation.

Reference to examples, embodiments, cases, instances, etc. are used to illustrate examples of different novel and improved techniques described further herein. The phrasing is not intended to limit the combination of different examples disclosed in different sections of this disclosure, unless otherwise specified. The terms “comprising,” “having,” and “including” are synonymous, unless the context dictates otherwise. The phrase “A and/or B” means (A), (B), or (A and B). The phrases “A/B” and “A or B” mean (A), (B), or (A and B), similar to the phrase “A and/or B.” For the purposes of the present disclosure, the phrase “at least one of A and B” means (A), (B), or (A and B). The description may use the phrases “in an embodiment,” “in embodiments,” “in some embodiments,” and/or “in various embodiments,” which may each refer to one or more of the same or different embodiments. Furthermore, the terms “comprising,” “including,” “having,” and the like, as used with respect to embodiments of the present disclosure, are synonymous.

Example embodiments may be described as a process depicted as a flowchart, a flow diagram, a data flow diagram, a structure diagram, or a block diagram. Although a flowchart may describe the operations as a sequential process, many of the operations may be performed in parallel, concurrently, or simultaneously. In addition, the order of the operations may be re-arranged, re-ordered, separated into additional operations, combined, or omitted altogether, unless explicitly stated otherwise. A process may be terminated when its operations are completed, but may also have additional steps not included in the description and/or FIG(S). A process may correspond to a method, a function, a procedure, a subroutine, a subprogram, and the like. When a process corresponds to a function, its termination may correspond to a return of the function to the calling function and/or the main function. A process may be implemented as program code, which may be stored by computer-readable storage media (e.g., locally or in the cloud), and when the program code is executed by one or more processors of a computer device/system, causes the computer device/system to perform the various operations of the process. Although the operations of a process may be described as being performed by one or more particular elements or devices discussed herein, other similar computing devices/systems (or components thereof) may operate such a process in a multitude of implementations, arrangements, and/or environments unless explicitly stated otherwise.

Example embodiments may be described in the general context of computer-executable instructions, such as program code, software modules, and/or functional processes, being executed by one or more examples of circuitry. The program code, software modules, and/or functional processes may include routines, programs, objects, components, data structures, etc., that perform particular tasks or implement particular data types. The program code, software modules, and/or functional processes discussed herein may be implemented using hardware in communication networks. For example, program code, software modules, and/or functional processes discussed herein may be implemented using hardware at network elements or control nodes.

As used herein, the term “circuitry” refers to, is part of, or includes hardware components such as an electronic circuit, a logic circuit, a processor (shared, dedicated, or group) and/or memory (shared, dedicated, or group), an Application Specific Integrated Circuit (ASIC), a field-programmable device (FPD), (for example, a field-programmable gate array (FPGA), a programmable logic device (PLD), a complex PLD (CPLD), a high-capacity PLD (HCPLD), a structured ASIC, or a programmable System on Chip (SoC)), digital signal processors (DSPs), etc., that are configured to provide the described functionality. In some embodiments, the circuitry may execute one or more software or firmware programs to provide at least some of the described functionality.

As used herein, the term “processor circuitry” may refer to, is part of, or includes circuitry capable of sequentially and automatically carrying out a sequence of arithmetic or logical operations; recording, storing, and/or transferring digital data. For example, code written using SQL, Python, etc., may be carried about by the processor circuitry. The term “processor circuitry” may refer to one or more application processors, one or more baseband processors, a physical central processing unit (CPU), a single-core processor, a dual-core processor, a triple-core processor, a quad-core processor, and/or any other device capable of executing or otherwise operating computer-executable instructions, such as program code, software modules, and/or functional processes. As used herein, the term “interface circuitry” may refer to, is part of, or includes circuitry providing for the exchange of information between two or more components or devices. The term “interface circuitry” may refer to one or more hardware interfaces (for example, buses, input/output (I/O) interfaces, peripheral component interfaces, network interface cards, and/or the like). As used herein, the terms “instantiate,” “instantiation,” and the like may refer to the creation of an instance, and an “instance” may refer to a concrete occurrence of an object, which may occur, for example, during execution of program code. Additionally, an “application instance” may be a realized software program executed in mobile edge host, which can provide service(s) to serve consumer(s).

As used herein, the term “computer device” (e.g., a user equipment) may describe any physical hardware device capable of sequentially and automatically carrying out a sequence of arithmetic or logical operations, equipped to record/store data on a machine readable medium, and transmit and receive data from one or more other devices in a communications network. A computer device may be considered synonymous to, and may hereafter be occasionally referred to, as a computer, computing platform, computing device, etc. The term “computer system” may include any type of interconnected electronic devices, computer devices, or components thereof. Additionally, the term “computer system” and/or “system” may refer to various components of a computer that are communicatively coupled with one another. Furthermore, the term “computer system” and/or “system” may refer to multiple computer devices and/or multiple computing systems that are communicatively coupled with one another and configured to share computing and/or networking resources. As used herein, the term “user equipment” or “UE” may refer to a device, such as a computer device, with radio communication capabilities and may describe a remote user of network resources in a communications network. The term “user equipment” or “UE” may be considered synonymous to, and may hereafter be occasionally referred to as client, mobile, mobile device, mobile terminal, user terminal, mobile unit, mobile station, mobile user, subscriber, user, remote station, access agent, user agent, receiver, radio equipment, reconfigurable radio equipment, reconfigurable mobile device, etc.

Examples of “computer devices”, “computer systems”, “UEs”, etc. may include cellular phones or smart phones, feature phones, tablet personal computers, wearable computing devices, an autonomous sensors, laptop computers, desktop personal computers, video game consoles, digital media players, handheld messaging devices, personal data assistants, an electronic book readers, augmented reality devices, server computer devices (e.g., stand-alone, rack-mounted, blade, etc.), cloud computing services/systems, network elements, in-vehicle infotainment (IVI), in-car entertainment (ICE) devices, an Instrument Cluster (IC), head-up display (HUD) devices, onboard diagnostic (OBD) devices, dashtop mobile equipment (DME), mobile data terminals (MDTs), Electronic Engine Management System (EEMS), electronic/engine control units (ECUs), electronic/engine control modules (ECMs), embedded systems, microcontrollers, control modules, engine management systems (EMS), networked or “smart” appliances, machine-type communications (MTC) devices, machine-to-machine (M2M), Internet of Things (IoT) devices, and/or any other like electronic devices.

As used herein, the term “network element” may be considered synonymous to and/or referred to as a networked computer, networking hardware, network equipment, router, switch, hub, bridge, radio network controller, radio access network device, gateway, server, and/or any other like device. The term “network element” may describe a physical computing device of a wired or wireless communication network and be configured to host a virtual machine. Furthermore, the term “network element” may describe equipment that provides radio baseband functions for data and/or voice connectivity between a network and one or more users. The term “network element” may be considered synonymous to and/or referred to as a “base station.” As used herein, the term “base station” may be considered synonymous to and/or referred to as a node B, an enhanced or evolved node B (eNB), next generation nodeB (gNB), base transceiver station (BTS), access point (AP), roadside unit (RSU), etc., and may describe equipment that provides the radio baseband functions for data and/or voice connectivity between a network and one or more users.

As used herein, the term “channel” may refer to any transmission medium, either tangible or intangible, which is used to communicate control information, data information, and/or any other type of signal. The term “channel” may be synonymous with and/or equivalent to “communications channel,” “data communications channel,” “transmission channel,” “data transmission channel,” “access channel,” “data access channel,” “link,” “data link,” “carrier,” “radiofrequency carrier,” and/or any other like term denoting a pathway or medium through which data is communicated. Additionally, the term “link” may refer to a connection between two devices through a Radio Access Technology (RAT) for the purpose of transmitting and receiving information.

As used herein, the term “computing resource”, “hardware resource”, etc., may refer to a physical or virtual device, a physical or virtual component within a computing environment, and/or physical or virtual component within a particular device, such as computer devices, mechanical devices, memory space, processor/CPU time and/or processor/CPU usage, processor and accelerator loads, hardware time or usage, electrical power, input/output operations, ports or network sockets, channel/link allocation, throughput, memory usage, storage, network, database and applications, and/or the like. As used herein, the term “network resource” may refer to computing resources that are accessible by computer devices via a communications network. The term “system resources” may refer to any kind of shared entities to provide services, and may include computing and/or network resources. System resources may be considered as a set of coherent functions, network data objects or services, accessible through a server where such system resources reside on a single host or multiple hosts and are clearly identifiable. Additionally, a “virtualized resource” may refer to compute, storage, and/or network resources provided by virtualization infrastructure to an application, such as a mobile edge application.

A communication system is described herein. The communication system may comprise one or network elements (e.g., server, network nodes, etc.), end-user devices (e.g., a UE), and/or sensor devices. The communication system may operate according to a centralized or decentralized software application, and/or one or more application-adjacent modules (e.g., software or hardware). The communication system may be interchangeable with application, unless otherwise specified. The communication system may include one or more hardware components (e.g., the network elements, server(s), UEs, etc.). The application may comprise one or more the modules (e.g., functions of the software instantiated virtually or in hardware specific instances); additionally, as described herein the application and the module(s) may be collectively referred to as the application, unless otherwise specified.

The application may run on one or more hardware components of the communication system, such as one or more servers (e.g., cloud based) that communicate with one or more client instances (e.g., UE, such as smartphones, tablets, laptops, desktops, etc.), using one or more communication technologies (e.g., wired, wireless, cellular LTE and/or NR, 802.11 Wi-Fi, etc.), via one or more networks (e.g., internet, local area network, etc.).

Additionally/alternatively, the application may run on each UE in the system (e.g., as the front end), and communicate with a one or more services running another instance of the application in a complimentary operation (e.g., as the back end). In one example, the client instances may be an interface presented on a UE for the cloud-based application (e.g., limited local processing is performed on the client side, such as the sending/receiving of signals using a transceiver and/or processor, generation of a display by a GPU, presentation of an interface by an operating system running on the processor operatively coupled with the transceiver, the displayed interface such as that displayed through a web browser). In another example, the client instances may be full applications running locally on the UE and communicating/generating/acquiring information/messages for another instance of the application running on one or more servers.

In one instance, the application may facilitate patient-centric care coordination and accountability. The application may be agnostic of a device operating system by being a web-enabled application deployed in the cloud (e.g., on a sever) and accessed through a web browser. The application may operate in real-time and may be always accessible. In some instances, the application may be inaccessible due to the maintenance of the application and/or related infrastructure. One or more interactions with the application by an end-user may be recorded (e.g., to a central repository or database) as each interaction occurs (e.g., in real-time). Each interaction may have one or more associated triggers which cause the performance of an action by the application based on configured instructions and conditions.

The application may receive on or more messages (e.g., using a protocol such as an http request) from a remote client instance. As referenced herein, a client instance may refer to computer hardware or software that accesses a service made available by a server as part of a client-server model of computer networks; this functionality may comprise of a request-response scheme. The requests may be interactions from one or more client instances. The interactions may be message/signal based as further described herein, and may comprise, for example, selecting an option, entering data, receiving sensor input, and/or the like. These requests may be sent to the back-end API (e.g., running on the server), which then may return a response message (e.g., related data, an indication of an update based on the original message). Additionally, each message may carry an authorization, and/or may be based upon a prior authorization (e.g., based on related permissions for a given session, as further described herein). For instance, when a client instance makes a request, the back-end (e.g., the application on the server) may determine if the client instance is allowed to have what it is requesting and what that data is actually comprised of; upon satisfying these one or more conditions, the application may then return the data and the client instance may present any number of controls.

The application may employ a notification module (e.g., lambda, referring to a serverless and/or application adjacent function that runs under the AWS lambda platform) that is responsible for creating notifications based on a configuration and/or related data (e.g., tasks and shifts). The application may employ additional functionality that is based on specific request messages initiated by a client instance.

The notification module may run periodically or in real-time, and select one or more data set(s) of tasks (e.g., medications and/or activities). Depending on the configuration (e.g., received through a configuration message from a UE), the notification module may create a notification and add the notification to a queue.

The notification module, and/or a communication module, may monitor for the creation of notifications in the queue. Depending on the nature and/or a parameter of the notification, a corresponding action may be triggered by the notification module and/or communication module, such as sending a message to the associated/specified client instances. The message may use a given communications protocol, such as email, SMS, or the like. The message may also be conveyed via an in-app communication displayed as pop-up dialog depending on the nature of the client instance.

The application may provide solutions to a myriad of communication-based problems, such as the efficient and accurate communication of information through a network to appropriate devices (e.g., client instances) based on a pre-established hierarchy of system wide privileges, authorizations, capability, and the like. In one instance, the application may address challenges of care coordination and accountability in one or more related aspects of care. In one aspect, for example, the application may be configured to manage team based communications for a number of client instances (e.g., one for each team members) of varying requirements, permissions, privileges, authorizations, need for information, and the like (e.g., where one team member may be designated as a professional and/or licensed, and another team member may be designated as informal such as an unpaid volunteers or other user defined by some familial or pre-established relationship). In one aspect, for example, the application may be configured to ensure all tasks related to a patient are organized, scheduled, performed, and reported accurately and on-time. In one aspect, for example, the application may be configured to maintain a longitudinal single source of truth that tracks all events logged by all users (e.g., caregivers) of the application. For example, across multiple users, there may be a verified source of information, which explicitly or inherently indicates where the information came from, as tracked by in a back-end operation or storage.

In some cases, in order to ensure accuracy of information describing an event, information related to that event may need to be recorded as close to the occurrence of the event as possible. This timeliness may ensure clarity and detail. However, the timely recording of information describing an event may be delayed by one or more other sources of interference. For example, a user may not be able to enter the information into the client instance immediately (e.g., there may be a need to perform another task, such as handwrite information in a chart, and/or or switch hardware related to accessing the client instance). Additionally, outsourcing the recordation of information to another human (e.g., such as manual transcription) may have issues with accuracy and/or timeliness.

Accordingly, there is a need for timeliness that only a communication system operating with a cloud-based application can provide. Further, the techniques and approaches described herein may further provide enhancements to existing technologies, such as those related to hardware efficiency (e.g., faster processing time, faster transmission time, faster intra-system communication time, lower power usage due to improved processing time and/or simplified processing methodologies, etc.) by implementing one or more techniques and/or approaches as described herein.

Generally, timeliness may have one or more related characteristics. Timeliness may improve ultimate end goals and provide efficiency improvements for an application, for example, if scheduled tasks are completed within a bounded timeframe of the task's scheduled time. Relatedly, information associated with that task may need to be disseminated to relevant constituents within a reasonable length of time after the state and/or disposition of a task(s) have changed (e.g., states and dispositions for definitions further described herein).

The application timeliness may be important because other related critical functions may be time dependent, such as the performance of calculations and sending response messages based on one or more parameters (e.g., set by an account owner and the actual interaction of participating users, such as caregivers). For example, if the application determines or receives input that a patient has a scheduled medication, the application may then interact with a connected smart bed to gently raise the position of the patient to sitting up so that they can be ready to take the medication.

The application may be configured to manage one or more users. The application, specifically, may carry out a management module. The management module may ensure that only the configured people have access to information; this may be accomplished my controlling access to information by allowing/approving a request for access to information and/or for modification of information based on the user or parameter of the user. The management module may coordinate schedules so that all a specific client instance (e.g., associated with a specific user with specific parameters related to the authorization of that user) may receive information related to tasks and other patent related responsibilities; this coordination may use one or more algorithms that ensures a task or patient related responsibility is allocated properly (e.g., in one case, a task may only need one user, so the application may determine which user, and my prevent more than one user being assigned the same task). The management module may enable and provide information at the same time to one or more users as appropriate in order to minimize errors and maintain consistency in the timeliness of the most up to date information (e.g., all users may need to know when some information has changed, which may be included in an update disseminated to all client instances based on a trigger). The management module may align the receiving and sending of information proportionally and appropriately to one or more users based one or more configured parameters of the user, such as classes and/or designations (e.g., authority, access rights, privilege, etc.).

In the application, there may be a plurality of users, and each user may belong to one or more classes, and have one or more designations. Each class and/or designation may have one or more privileges, authorizations, tasks, control, access rights, etc., within the application. For example, a user may be an account owner, which may have a specific class/designation related to an ultimate authority for a given patient. For example, a user may be a caregiver, which may be a user with a class/designation related to carrying out a treatment plan and/or who is responsible (e.g., receives and sends information related to) for one or more tasks associated with the welfare of the patient; this user may be further divided into formal and informal classes/designations. For example, a user may be an observer, who may have read only class/designations, limited in some cases depending upon further configuration, such as formal or informal.

For example, the application may restrict access to a given client instance to ensure that only authorized individuals can access the application (e.g., a patient record). In one case, a message may be sent from the client instance associated with an Account Owner that then configures the application to modify an existing user class/designation, and/or send a message to initiate a new client instance associated with a new user (e.g., an account owner has the ability to invite and remove team members) For example, a message may be received from a client instance associated with an account owner, where the message includes an address. The address may be used by the application to send an invitation message, and the invitation message may be sent to the address using the appropriate communication protocol. In one example, the invitation message may be an email, an SMS, a message through a third-party messaging platform, etc. The invitation message may include a hyperlink with an embedded one-time password. The application may receive a message initiated by the hyperlink, which may instruct the application to create a new user. The client instances associated with the account owner may receive a message indicating confirmation of the creation of the new user or failure of the new user, or a time-out of the invitation message (e.g., the hyperlink was not accessed within a time threshold). The application may receive a message from the client instance associated with the Account Owner regarding changing a parameter associated with any user as it relates to a patient (e.g., a message indicating a change of permissions for the new user). The application may send a message confirming that a change was made to access parameters of the user.

In one example, the access parameter for any given user may relate to permissions for information access and modification. There may be an access parameter that may change from “participating” to “observing” or from “observing” to “participating.” Participants may have limited write and full read access; they can complete tasks and enter data/text, but they cannot create new Tasks that populate the Task List (more later). Observers have read access only.

The application may utilize a Responsible Accountable Consulted and Informed (RACI) management framework based on instructions received from a client instance associated with the Account Owner, and messages received from client instances by one or more users associated with a care team. Table 1 provides an example of parameter settings associated with rights with the application for different users.

TABLE 1
Example of RACI Parameters

| Role | Responsible | Accountable | Consulted | Informed |
|---|---|---|---|---|
| Account Owner | No | Yes | No | No |
| Formal | Yes | No | No | No |
| participating | | | | |
| caregiver | | | | |
| Informal | Yes | No | No | No |
| participating | | | | |
| caregiver | | | | |
| Formal observer | No | No | Yes | No |
| Informal observer | No | No | No | Yes |

The application may have additional/alternative management frameworks, such as customizations. For example, the Patient may also be an Informal Participating Caregiver if they are still involved in their own care. For example, the Account Owner may be either an Informal Participating Caregiver or an Observer. For example, the Account Owner may also be a Patient. For example, the Account Owner may be a Patient and an Informal Participating Caregiver.

There may be one or more features for facilitating management of the care team members. An interface of the client instance, and/or the application may provide a “caregivers” tab that presents a list of all care team users. Each care team user may be able to send a message, and consequently upload a photograph and select an identifying color to customize the user display within the client instance and/or as a configuration of the application to then disseminate (e.g., send for display at a client instance) on an as-needed basis. For example, if a client instance sends a message requesting information regarding a given user, the application may send a related photo, and/or instructions to present the client instance in a configured color. Alternatively, the client instance may be configured to present the color based on the user log-in information. The client instance associated with the Account Owner may send a message including instructions to change permissions (e.g., as described herein), as well as instructions to create shifts for each care team user. A shift is a period of time during which user may be designated as responsible for tasks associated with the patient's care plan (e.g., responsible may mean that the client instance receives notifications related to the tasks, triggers functions on the UE of the client instance, like polling connected sensors for information, and or triggers the performance of specific functions of smart devices, like a raising/lowering a thermostat, a bed movement, medicine dispenser, etc.). For example, this designation may result in the application sending a message related to a task to a specific client instance associated with a user based on the information of a configured shift. For example, there may be a configuration of user X for a shift of Monday 8 AM-11 AM, and upon the application determining a message related to a task must be sent during this shift, the application may then send this message to user X.

The application may generate, and the client instance may present and/or access and/or request, a visual representation of “shifts” in a dashboard. The client instance associated with the Account Owner may send a message with instructions to the application to create shifts of varying lengths with start/end dates, set as indefinite by not entering an end date, and/or recurring dates. The application may include an error check function that triggers a message to the client instance, or via other means as described herein, associated with the Account Owner if a determination is made that shifts overlap. Alternatively, the application may include an error check to ensure there is at least partial overlap. The shifts, with corresponding assigned care team users and their selected colors, may be automatically overlayed against all created tasks in a master schedule.

An account owner may configure a time zone for all operations related to a patient. In one case, all users may be in the same time zone as the patient. In one case, some users may have different time zones, but the application may operate (e.g., all triggered responses such as notifications) on one time zone (e.g., of the patient).

Each task may have one or more related time parameters. For example, these time parameters (e.g., as set by a user) may include one or more of the following: start date and/or time; end date and/or time (e.g., a task without an end date makes the task indefinite); number of occurrences per given time unit (e.g., year, month, week, day, hour, unspecified, etc.) (e.g., as needed, daily, every other day, every three days, every week, etc.) (e.g., selection of “as needed” makes the task a “planned as needed task”); grace period, which is the window of time (e.g., in minutes) during which a task may be completed successfully; and/or, select reminder period, which is the window of time during which a reminder notification is triggered.

When a message is sent indicating a task is completed, that message may include the identity of the task and the time of fulfillment. The application may calculate the change in state and disposition of that task based on the time and the parameters specified for that task. The application may then send a response message to the client instance indicating the calculation for the task has been stored/recorded and/or causing the client instance to modify the display/appearance of that task accordingly.

As described herein, the accurate and timely completion of tasks and their accurate and timely reporting are critical to the application, and critical the application's ability to successfully manage and provide information regarding patient treatment plans, particularly those external to established healthcare information technology systems. For example, records related to medications may include time sensitive triggers. For example, records related to measurement information (e.g., such as blood sugar) must be requested/acquired/sent within the application periodically at specific time intervals, and if not, then other functions may be triggered to address the failure of that task. For example, a blood pressure or heart rate monitoring device may be operatively connected to a UE running a client instance, which may be able to facilitate the timely reporting of information to trigger one or more tasks. The reporting itself may be a task in some cases.

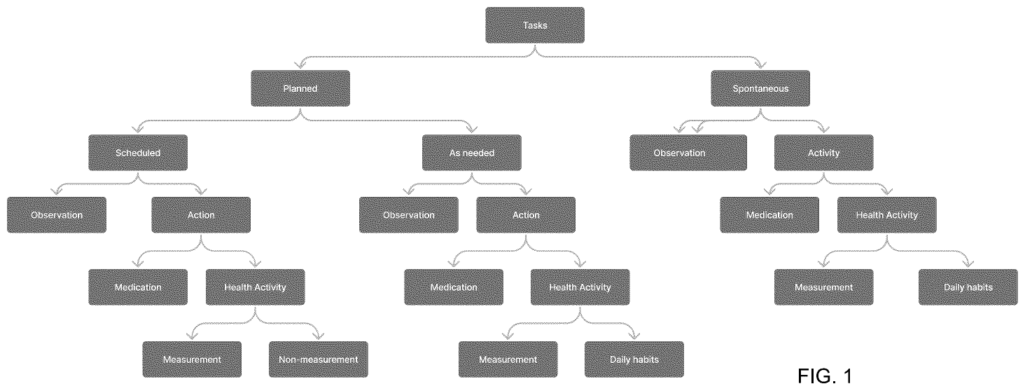

FIG. 1 illustrates an example of a task scheme.

Generally, the application may have a task module. The task module may be based on a configured task scheme. The task module may include one or more related functions in order to manage tasks. A task may include sending of a message, wherein the message may include the request of input from a client instance, the message includes instructions for displaying information via the client instance, and/or performing a related action (e.g., such as related to a connected device/sensor).

There may be one or more types of tasks. For example, there may be a planned task that has been previously configured in the application, where only a message from a client instance associated with the account owner has permission to read/write a configuration for a planned task. For example, there may be a spontaneous task that has not been previously configured in the application, where the application may receive a message associated with any caregiver user (e.g., that is a part of a designated team for a patient) that prompts the recording/reporting information related to the task. Further, unplanned tasks may take the form of sending a message, such as a message including information regarding observations, a message including information regarding a patient's body temperature, and/or a message including information regarding or a medication.

There may be one or more type of planned tasks. For example, there may be a scheduled task, which is a planned task that has been previously set to occur at specific date(s)/time(s); these may be one-time or recurring. For example, there may be an as needed task, which is a planned task that has not been previously set to occur at specific date(s)/time(s); these may also be known as “pro re nata” (PRN).

For any given task, there may be one or more type associated with the task. For example, there may be an observation type, which may relate to a message that requests or comprises information related to a qualitative description of the patient or circumstances around the patient. For example, there may be an action type, which may relate to a message that requests or comprises information related to the completion of a specific objective related to the patient's care.

For F any given action, there may be one or more associated objectives. For example, there may by a medication objective, which may relate to a message that requests or comprises information related to the administering of medication. For example, there may by a health activity objective, which may relate to a message that requests or comprises information related to all other aspects of the care plan other than medication. In some examples, there may be a field limited to expected range of a data type. For example, a field be limited to all weights possible for a human being, or limited to a range of numbers for recording a human temperature. For example, a field may be constrained in its numerical value to avoid error.

For any given activity/objective/task, there may be a related one or more information types. For example, one information type may be a measurement, which may relate to a message that requests or comprises information related to a health metric. The health metric may be manually entered into the client instance, or may be acquired directly or indirectly, as it is measured/assessed using an external device, the metrics related to, for example, blood sugar, body temperature, blood pressure, weight, etc. For example, one information type may be daily habits, which may relate to a message that requests or comprises information related to any health activity that is performed by the patient either with assistance from a caregiver or as directed/reminded by a caregiver that is related to health, such as eating, drinking water, sleeping, exercise, physical therapy, etc.

In some cases, a task may have one or more additional dimensions that add information to a task and are recorded as part of the record for each task in the application, such as: shift, which is period of time assigned to a caregiver during which a task occurs; user, which may be associated with a client instance that receives/sends a message related to the task; time, which includes when a message indicates that a task is completed as recorded in the application.

The tracking and calculation of time may be critical to the application. There may be a default state for any scheduled task, such as incomplete. The state may change based on one or more factors configured via a message from a client instance associated with the Account Owner. The factors may include one or more conditions, a time trigger (e.g., passing of configured amount of time, or a specific time), the time a task is actually completed as indicated in a message, and/or the time a message is received/sent that indicates completion of a task. The possible states of scheduled tasks may include, incomplete, complete, and/or expired. Additionally, the application may track information (e.g., receive/send messages to a client instance) related to a disposition of a task, as they relate to incompletion or completion. For example, the dispositions may be early, on-time, and late (e.g., a message may be send/received indicating to the application that a task is completed early). Depending on any factor/parameter/state associated with a task, there may be one or more related triggers causing the transmission of a message, the request of a message, and/or the performance of a function.

The application may include one or more critical time modules and/or functions. As such, certain time-related definitions may be important to the operation of the application and its components. For example, the current time may be the current local time of the end-user. For example, scheduled time may be the time at which a task in the treatment plan of a patient is scheduled to occur. For example, a grace period may be the amount of time identified by the end-user that establishes the time bound within which an event can occur and be considered “on-time”. For example, an incomplete task is a task that has not yet been completed but is not yet expired. For example, an incomplete early task is a task that has not yet been completed, is not expired, but now falls within the early disposition. For example, an early completed task is a task that has been completed early relative to its scheduled time and grace period. For example, a late incomplete task is a task that has not yet been completed, is not expired, but now falls within the late disposition. For example, a late completed task is a task that has been completed but late relative to its scheduled time and grace period. For example, an on-time incomplete task is a task that has not yet been completed, is not expired, but now falls within the on-time disposition. For example, an on-time completed task is a task that has been completed within its allotted grace period. For example, an expired task is a task that has not been completed and is now outside its allotted grace period.

The application may use one or more algorithms to determine a time as it relates to the performance of sending/receive a related message. In these algorithms, x=current time, t=scheduled time, and n=grace period.

For example, ff x<(t−2n) or x>(t+2n), default state= “incomplete”, no disposition. For example, If (t−2n)<=x<(t-n), default state= “early incomplete”, completion changes state to “early complete”. For example, if (t-n)<=x<=(t+n), default state= “on-time incomplete”, completion changes state to “on time complete”. For example, if (t+n)<x<=(t+2n), default state= “late incomplete”, completion changes state to “late complete”. For example, if (t+2n)<x, default state= “expired”, completion not possible.

All states and dispositions of tasks are recorded accordingly for each scheduled task in the application. These calculations trigger notifications to a configured one or more users (e.g., care team). Each notification, and related trigger, may has a specified communication type. For example, a communication type may be email, SMS, and/or a communication to a user specific, or user agnostic, client instance, as described herein.

As described herein, there are scheduled and as-needed tasks. As-needed tasks may not have an initial state because they are not scheduled. As-needed tasks, when completed, may automatically receive a state of “on-time complete”. As-needed tasks, when completed, may trigger a notification similar to and/or the same as scheduled tasks. Merging scheduled tasks that have set times with as-needed tasks that do not have set times may be done so that they may be executed within the same workflow.

TABLE 3
Example of a notification scheme

| | | | Triggered from | | |
|---|---|---|---|---|---|
| | | | main toolbar | | |
| Type of activity | Medication | Activity | (golden button) | Measurement | Frequency |
| Scheduled Medication | Yes | No | No | N/A | All except for |
| | | | | | “as needed” |
| Scheduled Non- | No | Yes | No | No measurement | All except for |
| Measurement Activity | | | | | “as needed” |
| Scheduled | No | Yes | No | Measurement | All except for |
| Measurement Activity | | | | | “as needed” |
| Scheduled | No | Yes | No | Observation to | All except for |
| Observation Activity | | | | text box | “as needed” |
| PRN Medication | Yes | No | Yes | N/A | As needed |
| PRN Non- | No | No | Yes | No measurement | As needed |
| Measurement Activity | | | | | |
| PRN Measurement | No | No | Yes | Measurement | As needed |
| Activity | | | | | |
| PRN Observation | No | No | Yes | Observation to | As needed |
| Activity | | | | text box | |
| PRN Health | No | No | Yes | Text box | N/A |
| Observation | | | | | |
| Patient Note | No | No | Yes | Text box | N/A |

One of the biggest challenges for capturing information related to tasks that are performed as part of a treatment plan is that conventional methods for recording information may be low-dimensional spaces. For example, a grid chart of a patient's activities may only contain three dimensions: the space itself (patient identity), the horizontal axis for the types of activities, and the vertical axis for time. Thus, a caregiver may indicate that activity x occurred at time y for patient z. Overlays of colors or insertions of symbols within each grid-square may add additional dimensionality such as the identity of the caregiver or whether the activity was late. However, there are limitations to the manner in which additional information can be added, and used, and there is also a need for more detailed information in order to enhance workflow, improve accuracy, and/or improve the time it takes to process a related function or data entry.

The data of the application (e.g., received, recorded, stored, sent, generated, related to tasks, related to functions, related to modules, etc.), as described herein, may be n-dimensional and may require high-dimensional spaces to encompass all available data and perform related operations/functions. The tension between expediency and ease of interpretation provided by low-dimensional spaces with the need for granularity and specificity provided by high-dimensional spaces is a significant challenge. For example, the need for tracking and organizing high-dimensional data spaces may be particularly important as it relates to patient care provided outside traditional healthcare settings.

Maintaining a coherent, longitudinal single-source of truth may be even more complicated when data types have an unequal number of dimensions. A particularly cumbersome example are tasks that are “spontaneous” and “as needed”. As defined earlier, a “spontaneous as needed task” is a task that is unplanned (e.g., not documented as a possibility in advance) and is unscheduled (e.g., only occurs as needed). As a result, this type of task cannot be placed within the same low-dimensional space that tracks “planned scheduled tasks” and “planned as needed tasks”, and consequently may have previously had limitations on being implemented in an overall application.

In an example, there may be a five-dimensional data point, including: Dimension 1 Patient Name, Dimension 2 Time of day, Dimension 3 Planned (really a binary condition); Dimension 4 Scheduled/as needed, and Dimension 5, Scheduled time. Table 4 shows how any given task may become infinitely more complex.

TABLE 4
Example of Task Dimension Complexity

| | | |
|---|---|---|
| Time 1 | Time 2 | Time X |

Planned Scheduled task 1 @ time X
Planned scheduled task N @ time X
Planned as needed task 1
Planned as needed task N

An unplanned event, by its nature, does not have “Planned” as a dimension; in other words, it does not yet exist because it has not yet been created. It in effect is missing a dimension and cannot be added to the matrix above. Unplanned tasks also by their nature can only be “as needed”.

One type of unplanned task may be an over-the-counter (OTC) medication that has not been taken before by the patient. For example, an unplanned task may be related to a specific antacid for very infrequent heartburn that the patient has never taken before. It is not planned as a task in the application because the patient has never taken it before. The patient has never taken it before because they have never really had heartburn severe enough to warrant any over-the-counter remedy. However, a user could have that OTC medication on hand when the patient happens to experience severe heartburn for the first time. The user then administers that antacid and reports the same via the client interface.

Another example of an unplanned task is a qualitative observation. An observation is a qualitative description entered into the application of something that the user has observed either about the patient or circumstances/situations related to the patient. The user may notice a change in behavior or mood that is worth inputting into the application. This type of information may be most useful when recorded accurately with date/time indicated (e.g., automatically or manually), possibly including one or more additional data points external to or integral to the input process (e.g., external might be the application gathering data such as the weather from a public data source, or integral data point may be a connected sensor, such as a temperature data point from a UE connected thermometer, or a picture taken by a UE). The Table 4, as an example, shows how this type of information may be challenging to easily be captured in low-dimensional space, and consequently, traditional systems may fail whereas the application described herein may enable faster processing times and the inclusion of additional data points not previously possible.

Planned as-needed tasks may be complicated in a low-dimensional space. Even though “planned as needed” tasks can be included in the matrix (e.g., perhaps with additional dimensions, such as color coding indicating that dimension), it may still be inefficient for recording purposes because it would require listing out in advance every single possible “as needed” task, which may be cumbersome and potentially disrupt workflow, cause confusion, and increase the probability of error.

The application described herein may handle tasks through a request→response scheme, and may include associated calculations as described herein. The application may automatically create the longitudinal single-source of truth record of all events associated with a patient. The information may be captured with all dimensions previously indicated, and have the capacity for additional dimensions that have not yet been anticipated. This ability may enable the processing of operations/functions/input/output that are more complex and faster than previously possible.

Furthermore, to support early intervention when treatment adherence falters, the application may receive a message from a client instance that includes one or more conditional summary notices, such as shown in Table 5.

TABLE 5
Conditional Summary Notices

| | |
|---|---|
| Text message | Email |

X medications missed in Y days
X activities missed in Y days
X events missed in Y days

These summary calculations are only possible based on the applications' longitudinal single-source of truth record of all events associated with the patient.

In some cases, the application may act as an integration hub for disparate data sources (e.g., generated as part of a treatment plan). For example, there may be: Self-reported information logged by a user who is a patient as well as other users (e.g., caregivers, doctors, healthcare workers, relatives, friends, etc.); connected IoT devices that generate biometric readings (e.g., blood pressure, blood sugar, body temperature, pulse oximeter, CPAP sleep monitors); API connection(s) to internet-enabled devices that generate health behavior data (MyFitnessPal, Peloton, Mirror, etc.); electronic health records platforms (e.g., MyChart); educational and just in time start of care guidelines and resources; (OBI) Outcomes based implementation data collection for continuously improved performance and learning; linked messaging applications (e.g., if in-application messaging is not implemented).

This data nexus may be organized around a specific record identifier (e.g., a patient's identification) and build upon the application's design of storing high-dimensional data centered around a specific case (the patient).

In addition to the disclosed examples, there may be other specific types of data, such as categorical, continuous, and/or text.

The application may be able to handle different data types simultaneously for analytics and predictive modeling. The handling of categorical and continuous data is available using a method for transforming disparate data types for modeling.

Text may be handled using Natural Language Processing (NLP) to transform phrases and sentences into coherent categorical variables. Sequenced parse tree (syntactic analysis) followed by semantic parsing and relational extraction would be employed to categorize and classify meaning; both categorical and continuous formats can be constructed from free-form text.

The data nexus may be an econometric panel from which specific types of analysis can be conducted to create discrete, novel data products. The panel may leverage known artificial intelligence methods as well as additional machine learning methods (supervised and semi-supervised learning, etc.). An econometric panel (also known as panel data) may be multi-dimensional data comprising observations over time. The two primary sub-sets of panels may be balanced and unbalanced. A balanced panel is one in which an observation is recorded for every panel member. An unbalanced panel is one in which at least one panel member has at least one observation missing. Of note, the application may function as both a balanced panel and an unbalanced panel which reflects the reality that not all patients will always have observations at every time interval.

The application may include modules for one or more of following: adherence proclivity, which is the likelihood a patient will adhere to therapy and approaches to increase this likelihood (e.g., through notifications, triggered user interactions, etc.); holistic health score, which may be a heuristic based on the integration of behavioral information (e.g., physical activity, health behaviors, nutrition, adherence, etc.) to assess the relative health and risk of specific outcomes for use in health pool risk management; diary panel/tracking mechanism for clinical trials, which may be a summary assessments of all activity during a clinical trial by the trial participant; user performance ratings (e.g., for a caregiver of the patient), which is a score/rating system for the performance of caregivers as part of a treatment plan; and/or, adjudication of family disputes over level of care provided, which may include a dispassionate and objective record/accounting of the relative effort of different family members in the care of a loved one, especially when disagreements over estates and wills are involved.

The application may include additional modules for data assessment, as might be used to trigger additional actions/tasks/notifications, as well as generate schedules and plans. For example, an assessment module (e.g., based on some degree of machine learning) may detect patterns within the data sets collected/generated by the application's (e.g., data nexus). For example, the data can be associated with a Schrödinger equation whose potential is determined by the data. The method described by Weinstein and Horn of Stanford Linear Accelerator may be used, which for a given set of data-points in some feature space may be associated with a solution. This may lead to good clustering solutions. Weinstein and Horn of Stanford Linear Accelerator approach may extend into a full-fledged dynamical scheme using a time-dependent Schrödinger equation.

In one case, the application may implement techniques (e.g., in conjunction with those described herein) described in the appendix (Technical description by Lawrence Choi) attached to this application.

In one case, a UE may need to register with a server in order to become a client instance of the application running on the server. For example, for the initial registration process between a UE and a server, there may be several steps to establish a secure and authenticated connection.

First, there may be a client initialization. The client initiates the registration process by sending a request to the server. This request typically includes basic information about the client and its intention to register.

Then, there may be a server check. The server checks its availability and responds to the client's request. If the server is available, it proceeds to the next step; otherwise, it may send an error message.

Then, there may be a handshake. To ensure secure communication, the server may challenge the client by sending a random nonce (a unique number used only once). The client responds to this challenge by encrypting the nonce with a pre-shared key or using a secure key exchange protocol.

Then there may be client validation. The server verifies the client's identity by decrypting the response and checking if it matches the expected value. If the client's identity is successfully verified, the server proceeds to the next step.

Then, there may be account creation. If the client is a new user, the server creates a new account and generates a unique identifier (such as a user ID or username) for the client. If the client is re-registering, the server may validate the provided credentials against its database.

Then, there may be token management. Upon successful identity verification and account creation/validation, the server generates a secure token (such as an access token or session token) and sends it to the client. This token serves as proof of the client's authentication and is used for subsequent interactions.

Then there may be client confirmation. The client receives the token and confirms the registration process. It may store the token securely for future use.

Then there may be session establishment. With the token in hand, the client can now establish a session with the server. This session allows the client to perform authenticated actions, and the server can associate the client's requests with their identity.

In some cases, depending on the application and security requirements, additional steps such as email verification, two-factor authentication, or other security measures may be included in the registration process.

In one example, the client instance may carry out one or more method steps in order to implement one or more of the features described herein. For each of the following steps there may be interactions between the client instance and the application (e.g., hosted on a server). The client instance may receive credential information from a user, which is then authenticated at the application. Authentication ensures that only authorized personnel can access patient data and record observations. Upon logging in, the client instance may present a dashboard (e.g., receive from the application or generate locally based on specific data points provided previously) that provides an overview of the users scheduled tasks, patient information, any relevant notifications, and a mechanism to enter information.

The user may select a specific patient from their schedule or search for the patient's profile. The patient profile includes essential information such as medical history, care plans, and previous observations. Alternatively, the client instance may only provide the patient information designated for that user at that time. The user may input observations about the patient's vital signs, symptoms, and overall condition. This may involve manual input or, in the case UE connected sensors like a Bluetooth thermometer, automatic data acquisition. The application may prompt the worker to use specific sensors and guide them through the process.

If the patient requires monitoring using sensors, the client instance may control the UE to interact with these sensors. For instance, a Bluetooth thermometer may measure the patient's temperature. The application interfaces with the sensor, collects the data, and associates it with the patient's record.

The application monitors the patient's care plan and triggers notifications for important events, such as medication reminders, scheduled check-ins, or critical changes in the patient's condition. The client instance receives these notifications in real-time to ensure timely and appropriate responses.

The user may access the patient's care plan through the client instance. This plan outlines the specific tasks, medications, and procedures that need attention. The user may update the care plan based on the patient's evolving needs and conditions.

The application may include communication features, allowing the healthcare worker to collaborate with other members of the patient care team (e.g., users of varying privileges). This may include messaging, notes, and updates to ensure everyone involved in the patient's care is informed.

The application in conjunction with user input may manage schedules, including appointment details, travel routes, and coverage. The scheduling system may integrate with the other backend systems to ensure accuracy and coordination (e.g., a third-party billing, email, or calendar system, etc.).

To maintain data integrity, the application synchronizes recorded observations, care plan updates, and other relevant information across client instances, and across one or more servers (in the case of a decentralized server group). This ensures that all authorized users have access to the most recent and accurate patient data.

In one or more examples as described herein, additional sensors may be utilized to enhance the application and client instance functionality. For example, there may one or more of the following that can connect independently to the application (e.g., an Internet connected device, via WiFi for example), or can connect to the UE to interact with the client instance, which in turn communications with the application: a bluetooth Thermometer (e.g., measures body temperature and can be useful for detecting fever, a common symptom of many illnesses; Pulse Oximeter (e.g., measures the oxygen saturation level in the blood and the heart rate. This sensor is often used to monitor respiratory conditions); Blood Pressure Monitor (e.g., Connects to the iPhone to measure blood pressure, providing crucial data for cardiovascular health monitoring); Electrocardiogram (ECG or EKG) Monitor (e.g., Captures the electrical activity of the heart, helping to diagnose various heart conditions and irregularities); Blood Glucose Monitor (e.g., Measures blood glucose levels, essential for managing diabetes. Data can be logged and shared with healthcare providers); Spirometer (e.g., Measures lung function by assessing the volume and flow of inhaled and exhaled air, which is useful for monitoring respiratory health, especially for patients with conditions like asthma or chronic obstructive pulmonary disease (COPD)); Activity Trackers (e.g., built-in accelerometers and gyroscopes in the UE can be used to monitor physical activity, steps taken, and estimate energy expenditure); Smart Scales (e.g., Measures body weight, body fat percentage, and other metrics, where some smart scales can sync data directly with health a client instance on a UE, or can connect to the application residing on a server via WiFi); Sleep Trackers (e.g., Devices that monitor sleep patterns by tracking movements and sleep cycles, where this information can provide insights into sleep quality); Glucometer (e.g., Measures blood glucose levels for individuals with diabetes. The iPhone application can store and analyze historical data to track trends); Skin Temperature Sensors (e.g., These sensors can monitor skin temperature variations, which might be relevant in certain medical conditions or during fever monitoring); Pregnancy Monitors (e.g., Specialized sensors for monitoring fetal heart rate and other parameters during pregnancy. Some devices can connect to an iPhone for data visualization and tracking); Smart Inhalers (e.g., Connected inhalers can track medication usage for individuals with respiratory conditions, helping to ensure adherence to treatment plans); Continuous Glucose Monitors (CGM) (e.g., CGMs provide real-time glucose readings for individuals with diabetes. Data can be transmitted to the iPhone for constant monitoring and analysis); and/or, Wearable Electroencephalogram (EEG) (e.g., some EEG devices are designed as wearables, measuring brain activity and providing insights into neurological health).

The application may perform one or more functions with respect to data management.

For example, the application may manage data representation, where it may define the structure of the data to accommodate multiple dimensions. This could be achieved using arrays, matrices, or more complex data structures, depending on the nature of the data.

For example, the application may manage database or persistent storage, where if the multi-dimensional data is extensive or needs to be stored for future use, a database or other forms of persistent storage may be used. Core Data, SQLite, or other local storage mechanisms may be employed to manage and retrieve data efficiently.

For example, the application may manage User Interface (UI) design that allows users to interact with the multi-dimensional data easily. This may involve graphical representations, charts, tables, or other visualizations that can display data across various dimensions.

For example, the application may manage navigation through dimensions, by implementing mechanisms for users to navigate through different dimensions of the data. This could involve sliders, dropdowns, or other UI elements that allow users to select and focus on specific dimensions or ranges within those dimensions.

For example, the application may manage filtering and sorting, and provide options for users to filter and sort data based on different dimensions. This enhances the user's ability to analyze and interpret the information in the multi-dimensional space effectively.

For example, the application may manage data aggregation and summarization, and allow users to aggregate or summarize data along different dimensions (e.g., possibly using advanced AIs such as large language models, e.g., ChatGPT, etc.). This could involve calculating averages, totals, or other statistical measures based on user-defined dimensions.

For example, the application may manage real-time updates, where, if the data in the multi-dimensional space is dynamic and subject to change, implement mechanisms for real-time updates that enable data integrity. This ensures that users receive the latest information as it becomes available.

For example, the application may manage data synchronization, where if the application is connected to a server or cloud-based storage, implement data synchronization mechanisms to keep the data consistent across different devices. This is crucial for applications that involve collaboration or data sharing.

For example, the application may manage data security, implement security measures to ensure the confidentiality and integrity of the multi-dimensional data, especially if it involves sensitive or private information.

For example, the application may manage performance optimization, in order to optimize data retrieval and manipulation operations to ensure smooth performance of the application and the client instance, especially when dealing with large datasets or complex computations in multi-dimensional space.

For example, the application may manage error handling, and implement robust error-handling mechanisms to address issues such as missing data, incorrect inputs, or other potential problems that may arise during data management operations.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random access memory (RAM), a register, cache memory, semiconductor memory devices, magnetic media such as internal hard disks and removable disks, magneto-optical media, and optical media such as CD-ROM disks, and digital versatile disks (DVDs). A processor in association with software may be used to implement a radio frequency transceiver for use in a UE, terminal, base station, RNC, or any host computer.

What is claimed:

1. A computer-implemented method for managing healthcare services, the method comprising:
receiving authentication details from a user via a mobile application;
verifying the identity of the user based on the received authentication details;
providing the user with access to healthcare data associated with one or more patients;
displaying the healthcare data on a user interface of the mobile application;
receiving healthcare indicators recorded by the user via the mobile application;
updating the healthcare data based on the received healthcare indicators; and
synchronizing the updated healthcare data with a central data repository.

2. The method of claim 1, further comprising:
initiating one or more connected health sensors by the user via the mobile application;
receiving health sensor data from the initiated connected health sensors; and
updating the healthcare data based on the received health sensor data.

3. The method of claim 1, further comprising:
triggering one or more alerts based on events associated with a healthcare plan of a patient; and
delivering the triggered alerts to the healthcare professional via the mobile application.

4. The method of claim 1, further comprising:
providing the user with access to a healthcare plan of a patient via the mobile application;
receiving updates to the healthcare plan from the user via the mobile application; and
updating the healthcare plan based on the received updates.

5. The method of claim 1, further comprising:
facilitating collaboration between the user and other users via collaboration tools provided in the mobile application.

6. The method of claim 1, further comprising:
providing the user with access to an appointment management system via the mobile application;
receiving updates to a schedule of the user via the mobile application; and
updating the schedule based on the received updates.

7. The method of claim 2, wherein the connected health sensors include a connected thermometer.

8. The method of claim 2, wherein the health sensor data includes temperature data.

9. The method of claim 3, wherein the alerts are triggered based on medication reminders, scheduled check-ins, or critical changes in a patient's condition.

10. The method of claim 4, wherein the healthcare plan includes specific tasks, medications, and procedures that need attention.

11. The method of claim 5, wherein the collaboration tools include messaging, notes, and updates.

12. The method of claim 6, wherein the appointment management system integrates with an organization's backend system.

13. A system for managing healthcare services, the system comprising:
one or more processors;
a memory storing instructions that, when executed by the one or more processors, cause the system to:
receive authentication details from a user via a mobile application;
verify the identity of the user based on the received authentication details;
provide the user with access to healthcare data associated with one or more patients;
display the healthcare data on a user interface of the mobile application;
receive healthcare indicators recorded by the user via the mobile application;
update the healthcare data based on the received healthcare indicators; and
synchronize the updated healthcare data with a central data repository.

14. The system of claim 13, wherein the instructions further cause the system to:
initiate one or more connected health sensors by the user via the mobile application;
receive health sensor data from the initiated connected health sensors; and
update the healthcare data based on the received health sensor data.

15. The system of claim 13, wherein the instructions further cause the system to:
trigger one or more alerts based on events associated with a healthcare plan of a patient; and
deliver the triggered alerts to the user via the mobile application.

16. The system of claim 13, wherein the instructions further cause the system to:
provide the user with access to a healthcare plan of a patient via the mobile application;
receive updates to the healthcare plan from the user via the mobile application; and
update the healthcare plan based on the received updates.

17. The system of claim 13, wherein the instructions further cause the system to:
facilitate collaboration between user and a healthcare team via collaboration tools provided in the mobile application.

18. The system of claim 13, wherein the instructions further cause the system to:
provide the user with access to an appointment management system via the mobile application;
receive updates to a schedule of the user via the mobile application; and
update the schedule based on the received updates.

19. The system of claim 14, wherein the connected health sensors include a connected thermometer.

20. The system of claim 14, wherein the health sensor data includes temperature data.
